(19) United States
(12) Patent Application Publication
(10) Pub. No.: US 2017/0128662 A1
(43) Pub. Date: May 11, 2017

(54) PACKAGING UNIT FOR SINGLE MEDICAL DEVICE

(71) Applicants: BAXALTA INCORPORATED, Bannockburn, IL (US); BAXALTA GMBH, Glattpark(Opfikon) (CH)

(72) Inventors: Nicholas John Schill, Johnsburg, IL (US); Patrick John Ryan, Buffalo Grove, IL (US); Stephan Delville, Lessines (BE); Ibrahim Cherkani, Ath (BE)

(21) Appl. No.: 14/934,784

(22) Filed: Nov. 6, 2015

(51) Int. Cl. A61M 5/00; B65D 77/22

(52) U.S. Cl. CPC A61M 5/002; B65D 77/22

(57) ABSTRACT

A packaging unit is provided for accommodating a syringe having a barrel for slidingly receiving a plunger at one end, and having an opposite needle end, and includes a housing defining a chamber for receiving the syringe. Included in the housing are a first cavity for receiving the syringe plunger, and having a first set of side walls for directly engaging a head portion of the plunger without engaging a middle portion of the plunger; a second cavity for receiving the syringe barrel, and having a set of upper walls for directly engaging an upper surface of a flange portion of the barrel; and a third cavity for receiving the needle end of the syringe, and having a second set of side walls for directly engaging a cover for enclosing the needle end. The second cavity is in fluid communication with the first and third cavities.

### BACKGROUND

The present disclosure generally relates to packaging units used for protecting medical devices, and more specifically to a packaging tray configured for protecting a syringe during shipping and handling.

For pharmaceutical products, packaging is an important aspect of an overall product design, because a medical device transported in the packaging tray is easily damaged during shipping and handling when the medical device is improperly secured within the tray. Typically, the medical device is prefilled with a medicinal substance for convenience. However, depending on the particular medicinal substance and the intended clinical indication, the plunger may be located in different positions, even though the same device or syringe is located in the package. Thus, it is important to accommodate various plunger positions in a single package, and to protect the fragile device, such as a syringe, against damage or unwanted movement of the plunger from external impacts.

On the other hand, in certain situations, a user may need to have immediate access to the pre-filled syringe when the medicinal substance is going to be promptly administered. In conventional packaging units, when removing the syringe from the packaging tray, if the syringe is secured in a complicated package, the user may experience difficulties and thus be unable to timely use the syringe. In this case, the syringe may be partially protected from undesirable movement, but it is challenging and difficult to quickly remove the syringe from the tray when needed. Further, during rushed handling by the user, removing the syringe from a complicated package can cause structural damage to the syringe, and untimely release of the medicinal substance. In some cases, pulling a plunger head prematurely from the packaging unit breaks a seal between a plunger and a syringe barrel, causing leakage of a medicinal fluid or substance from the barrel, or compromises the sterility of the medicinal fluid.

Thus, there is a need for developing an improved packaging unit that provides an enhanced storage arrangement for the medical device in a simpler and more reliable manner.

### SUMMARY

The present disclosure is directed to a molded packaging tray or unit for protecting a medical device, such as a syringe, during shipping and handling. An important aspect of the present packaging unit is that a plunger head of the syringe is movable in an upper cavity area of the packaging unit for accommodating the same syringe being used to deliver a variety of medicinal compounds in a variety of volumes. However, the plunger head is also tightly surrounded by side walls of the present tray which are dimensioned to deter a user from grasping the plunger head. Thus, the plunger head is securely held within the cavity area such that the plunger head is not accessible to the user until the syringe is removed from the packaging unit. In this configuration, the user is compelled to remove the syringe by holding the syringe barrel first to avoid unwanted breakage of the seal between the plunger and the syringe barrel.

Another important aspect of the present packaging unit is that two gripping hemispheres protrude into a main channel or chamber from side walls for holding the syringe barrel. These protrusions provide a friction fit between the tray and syringe for preventing unwanted movement of the syringe. Also, the present tray is constructed and arranged to provide a secure fit at a needle end of the syringe, which includes a needle protector (or cover) and an adjacent portion of the barrel. This design deters the user from attempting to remove the syringe from the packaging unit via grasping the needle protector. Further, a middle cavity area of the present tray is large and spacious enough to encourage the user to grasp the syringe barrel at this location for properly pulling the syringe from the tray.

Also, a bottom surface or floor of the packaging unit is specifically configured to follow a contour of the syringe. In this configuration, a first bottom surface profile is provided such that the needle protector and the lower portion of the barrel are supported by the tray in a cradled position, and a second bottom surface profile is provided such that an extra grasping space is formed under the syringe barrel, corresponding to the middle “grasping” cavity area. This “grasping” cavity area allows the user to safely and quickly remove the syringe from the packaging unit without causing structural damage.

In one embodiment, a packaging unit is provided for accommodating a syringe having a barrel configured for slidingly receiving a plunger at one end, and having an opposite needle end, and includes a housing defining a chamber configured for receiving the syringe. Included in the housing are a first cavity configured for receiving the syringe plunger, and having a first set of side walls for directly engaging a head portion of the plunger without engaging a middle portion of the plunger; a second cavity configured for receiving the syringe barrel, and having a set of upper walls for directly engaging an upper surface of a flange portion of the barrel; and a third cavity configured for receiving the needle end of the syringe, and having a second set of side walls for directly engaging a cover configured for enclosing the needle end. The second cavity is in fluid communication with the first cavity at one end, and at an opposite end with the third cavity.

In another embodiment, a packaging unit is provided for accommodating a syringe having a barrel configured for slidingly receiving a plunger at one end, and having an opposite needle end, and includes a housing defining a chamber configured for receiving the syringe. Included in the housing are a first cavity configured for receiving the syringe plunger, and having a first set of side walls for directly engaging a head portion of the plunger without engaging a middle portion of the plunger; a second cavity configured for receiving the syringe barrel, and having a set of upper walls for directly engaging an upper surface of a flange portion of the barrel; and a third cavity configured for receiving the needle end of the syringe, and having a second set of side walls for directly engaging a cover configured for enclosing the needle end. The second cavity is in fluid communication with the first cavity at one end, and at an opposite end with the third cavity, and a bottom surface of the housing has a first profile following the contour of the head portion of the plunger and the flange portion of the barrel, and a second profile following the contour of the needle end of the syringe and the needle end.

In yet another embodiment, a packaging unit is provided for accommodating a syringe having a barrel configured for slidingly receiving a plunger at one end, and having an opposite needle end, and includes a housing defining a chamber configured for receiving the syringe. Included in the housing are a first cavity configured for receiving the syringe plunger, and having a first set of side walls for directly engaging a head portion of the plunger; a second cavity configured for receiving the syringe barrel, and having a set of upper walls for directly engaging an upper surface of a flange portion of the barrel; and a third cavity configured for receiving the needle end of the syringe, and having a second set of side walls for directly engaging a cover configured for enclosing the needle end. The second cavity is in fluid communication with the first cavity at one end, and at an opposite end with the third cavity, such that a volume of the second cavity is larger than that of each of the first cavity and the third cavity, and at least one side protrusion is provided in the second cavity for directly engaging an outer surface of the syringe for providing a friction fit between the housing and the syringe.

The foregoing and other aspects and features of the disclosure will become apparent to those of reasonable skill in the art from the following detailed description, as considered in conjunction with the accompanying drawings.

### BRIEF DESCRIPTION OF THE DRAWINGS

FIG. 1 is a perspective view of the present packaging unit, featuring an exemplary syringe held in a housing of the present packaging unit;

FIG. 2 is a plan view of the packaging unit of FIG. 1;

FIG. 3 is a cross-section of the packaging unit taken along the line 3-3 of FIG. 2 and in the direction generally indicated;

FIG. 4 is a cross-section of the packaging unit taken along the line 4-4 of FIG. 2 and in the direction generally indicated;

FIG. 5 is a cross-section of the packaging unit taken along the line 5-5 of FIG. 2 and in the direction generally indicated;

FIG. 6 is a cross-section of the packaging unit taken along the line 6-6 of FIG. 2 and in the direction generally indicated; and

FIG. 7 is a partial, enlarged view of a circled portion A of the packaging unit of FIG. 1.

### DETAILED DESCRIPTION

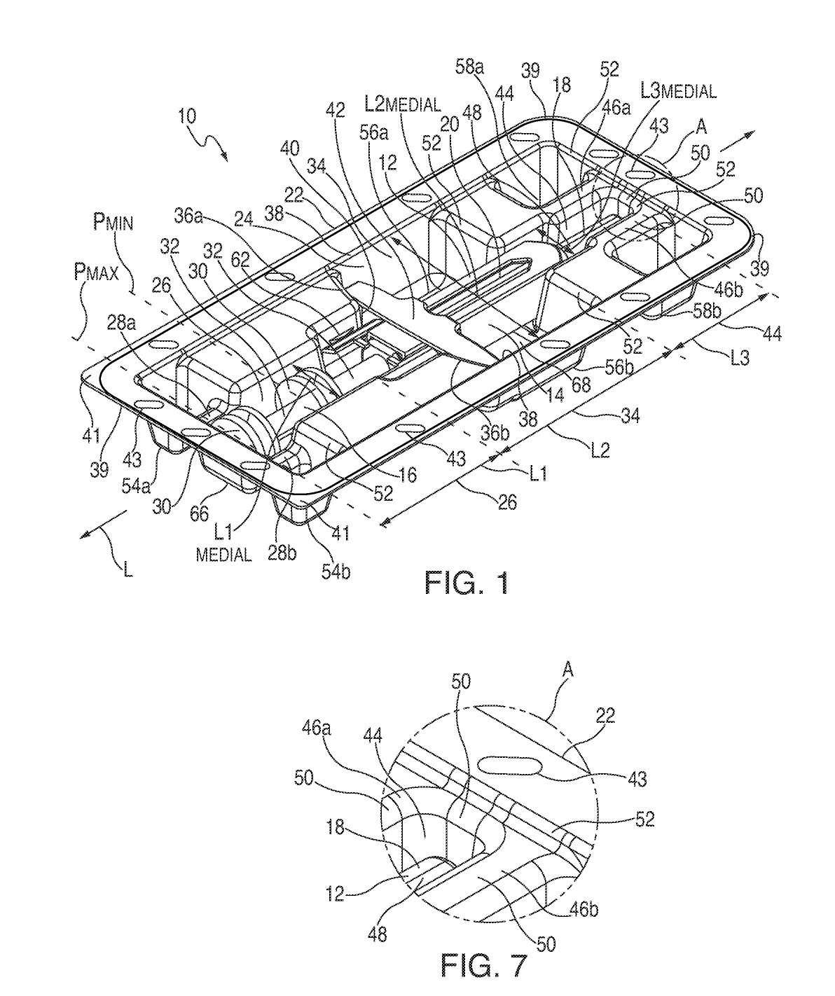

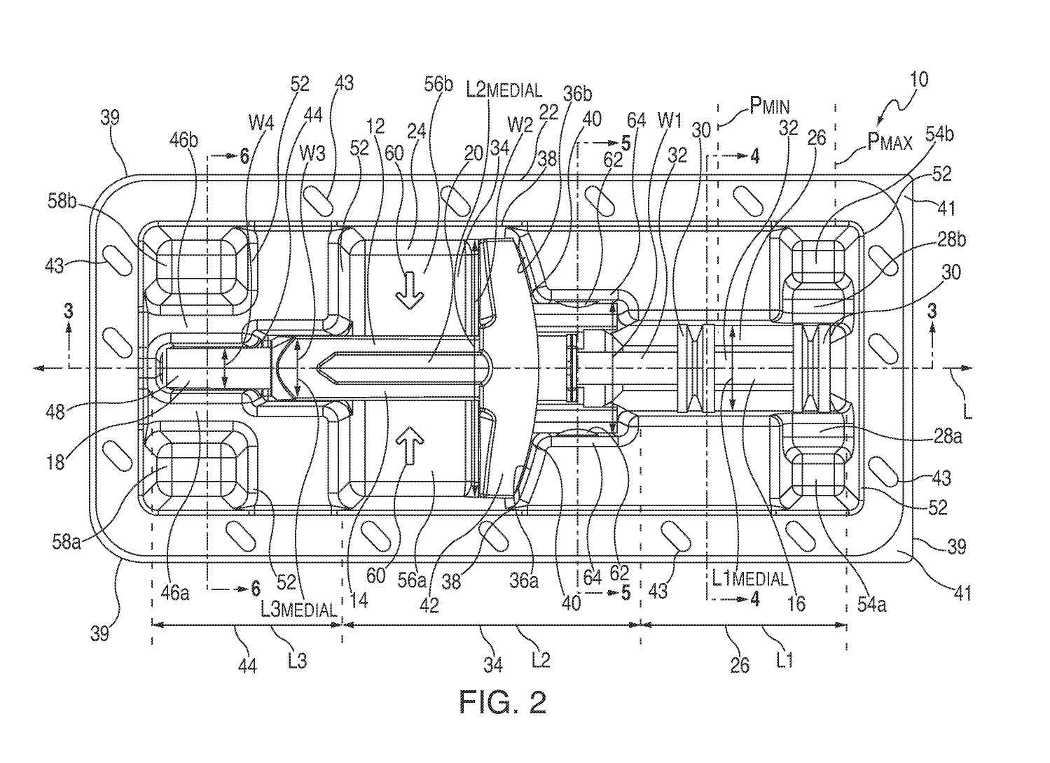

Referring now to FIGS. 1 and 2, the present packaging unit is generally designated 10 and is designed to accommodate a syringe 12 having a barrel 14 configured for slidingly receiving a plunger 16 at one end, and having an opposite needle end 18. An exemplary syringe 12 includes, but is not limited to, an auto-injector and a pre-filled disposable syringe. Typically, the syringe 12 has an inspection window 20 on the barrel 14 for viewing a medicinal fluid or substance stored in the barrel.

An important aspect of the present packaging unit 10 is that a housing 22 of the packaging unit defines a chamber 24 configured for receiving the syringe 12, and includes three particular cavity areas in the chamber. Specifically, a first cavity 26 is configured for receiving the syringe plunger 16, and having a first set of side walls 28a, 28b for directly engaging a head portion 30 of the plunger without engaging a middle portion 32 of the plunger. Since only the head portion 30 of the plunger 16 is engaged by the side walls 28a, 28b, the plunger is selectively displaceable relative to the barrel 14 within the first cavity 26. In this configuration, an amount of the medicinal substance stored in the barrel 14 is readily adjusted as desired.

In a preferred embodiment, the first cavity 26 is configured for accommodating a plurality of displacement positions for the syringe plunger 16 relative to the syringe barrel 14. For example, when the syringe 12 holds a maximum amount of medicinal substance, the head portion 30 of the plunger 16 is disposed at a maximum extendable position P_{MAX}. However, when the syringe 12 holds a minimum amount of medicinal substance, the head portion 30 of the plunger 16 is disposed at a minimum extendable position P_{MIN}. As a result, the head portion 30 is selectively positioned in the first cavity 26 of the packaging unit 10 for accommodating a reciprocating action of the plunger 16. It is advantageous that the same packaging unit 10 is used for storing the syringe 12 that facilitates various medical therapies involving different amounts of medicinal substance.

It is contemplated that the present packaging unit 10 has a second cavity 34 configured for receiving the syringe barrel 14, and the second cavity includes a set of curved or slightly arcuate upper or support walls 36a, 36b extending from a peripheral edge 38 of the housing 22 for directly and complementarily engaging an upper surface 40 of a flange portion 42 of the barrel 14. Further, a third cavity 44 is provided in the housing 22 for receiving the needle end 18 of the syringe 12, and the third cavity includes a second set of side walls 46a, 46b for directly engaging a cover or protector 48 of the syringe 12 configured for enclosing the needle end 18. As shown in FIGS. 1 and 2, the second cavity 34 is in fluid communication with the first cavity 26 at one end, and at an opposite end with the third cavity 44, thereby defining the chamber 24 of the housing 22.

Along the peripheral edge 38 of the housing 22, a turned-down flange 39 is provided around the outer circumference of the housing 22 at a predetermined width, such that a protective plastic cover or film (not shown) is removably attached to the turned-down flange for enclosing the chamber 24 of the housing using an adhesive or other adhering materials. It is preferred that at least one corner of the flange 39 forms an opening tab 41 for easy access to the plastic film enclosing the chamber 24. It is also contemplated that the flange 39 includes a plurality of negative or positive slots or textures 43 to readily break a sealing surface between the film and the flange. While the slots 43 having a diagonal formation on the flange 39 are shown, other suitable configurations, such as curved or coiled grooves or protrusions, are also contemplated to suit the application.

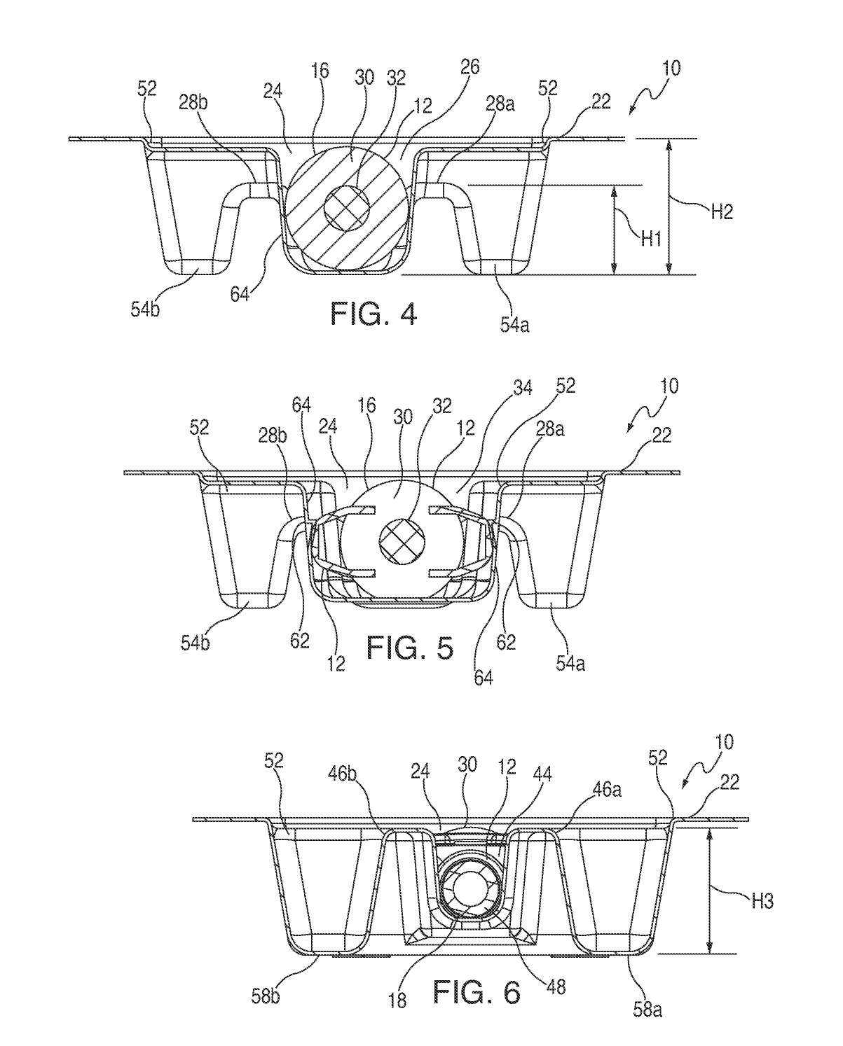

Referring now to FIGS. 1, 4, 6, and 7, another feature of the present packaging unit 10 is that a height H1 (FIG. 4) of the first set of side walls 28a, 28b in the first cavity 26 is shorter than a height H2 (FIG. 4) of the chamber 24 of the housing 22. Further, the height H1 (FIG. 4) of the first set of side walls 28a, 28b in the first cavity 26 is shorter than a height H3 (FIG. 6) of the second set of side walls 46a, 46b in the third cavity 44. In this configuration, the cover 48 protecting the fragile needle end 18 is fully and securely enclosed and held by the chamber 24. It is also contemplated that while FIGS. 4 and 6 show that the height H1 is shorter than the height H3, both heights H1, H3 can have the same height. Other various height arrangements of the side walls 28a, 28b, 46a, 46b are contemplated to suit different applications.

Further, as illustrated in FIGS. 1 and 7, surrounding side walls 50 including the second set of side walls 46a, 46b in the third cavity 44 firmly and entirely enclose the cover 48 of the syringe 12 to prevent horizontal syringe movement. It is preferred that an upper peripheral edge 52 of each of the first, second, and cavities 26, 34, 44 has a rounded or curved corner to facilitate easy insertion of the syringe 12 into the chamber 24 of the housing 22.

Returning now to FIGS. 1 and 2, a pair of first feet 54a, 54b is disposed adjacent a corresponding one of the first set of side walls 28a, 28b in the first cavity 26. A pair of second feet 56a, 56b is disposed in the second cavity 34, and a pair of third feet 58a, 58b is disposed adjacent a corresponding one of the second side walls 46a, 46b in the third cavity 44. While a square-shaped foot is shown for illustration purposes, other geometric shapes, such as cylindrical and conical constructions, are also contemplated. It is also contemplated that each pair of corresponding feet 54a and 54b, 56a and 56b, 58a and 58b can be integrally formed as one foot to suit the application.

In a preferred embodiment, a volume of the second cavity 34 is larger than that of each of the first cavity 26 and the third cavity 44. Further, an axial length L2 of the second cavity 34 is longer than the axial length L1 of the first cavity 26 and the axial length L3 of the third cavity 44 relative to a longitudinal axis L of the housing 22. It is also contemplated that a medial length L2_{MEDIAL} of the second cavity 34 transverse to the longitudinal axis L of the housing 22 is the longest relative to the medial length L1_{MEDIAL} of the first cavity 26 and the medial length L3_{MEDIAL} of the third cavity 44. Also, as illustrated in FIG. 2, the second cavity 34 has a graphical indicator 60 designed for informing a user of a proper grasping method of the syringe 12 for removal from the housing 22. While an arrow is shown as the preferred indicator 60, other shapes are contemplated. Also, other suitable indicators, such as recycle signs and other identifiers, are contemplated to suit the application.

Referring now to FIGS. 1, 2 and 5, the second cavity 34 has at least one protrusion 62 (FIG. 5) for directly engaging an outer surface of the syringe 12 for providing a friction fit between the housing 22 and the syringe. In a preferred embodiment, the protrusion 62 has a hemispherical shape, and protrudes into the second cavity 34 from side walls 64 of the housing 22. Other suitable shapes of the protrusion 62, such as triangular, conical, and trapezoidal shapes, are contemplated depending on different applications.

Another friction fit is similarly provided in the third cavity 44. Specifically, a width of the third cavity 44 is slightly narrower than the width of the cover 48 of the needle end 18 for providing the friction fit between the housing 22 and the syringe 12. Thus, the third cavity 44 is configured for tightly receiving the needle end 18 of the syringe 12 and the cover 48 of the needle end.

Another important configuration of the present packaging unit 10 is that the second and third cavities 34, 44 have at least two different widths relative to the longitudinal axis L of the housing 22. Specifically, a first or upper width W1 of the second cavity 34 is shorter than a second or lower width W2 of the second cavity, both the first and second widths being transverse to the longitudinal axis L of the housing 22. The first width W1 is a distance between the side walls 64 of the housing 22 above the flange portion 42 of the syringe 12, and the second width W2 is the distance between the side walls of the housing towards the needle end 18 relative to the flange portion.

Similarly, a third or barrel width W3 of the third cavity 44 is longer than a fourth or needle cover width W4 of the third cavity, both the third and fourth widths being transverse to the longitudinal axis L of the housing 22. The third width W3 is the distance between the side walls 64 of the housing 22 adjacent a lower end of the barrel 14, and the fourth width W4 is the distance between the side walls of the housing adjacent the cover 48.

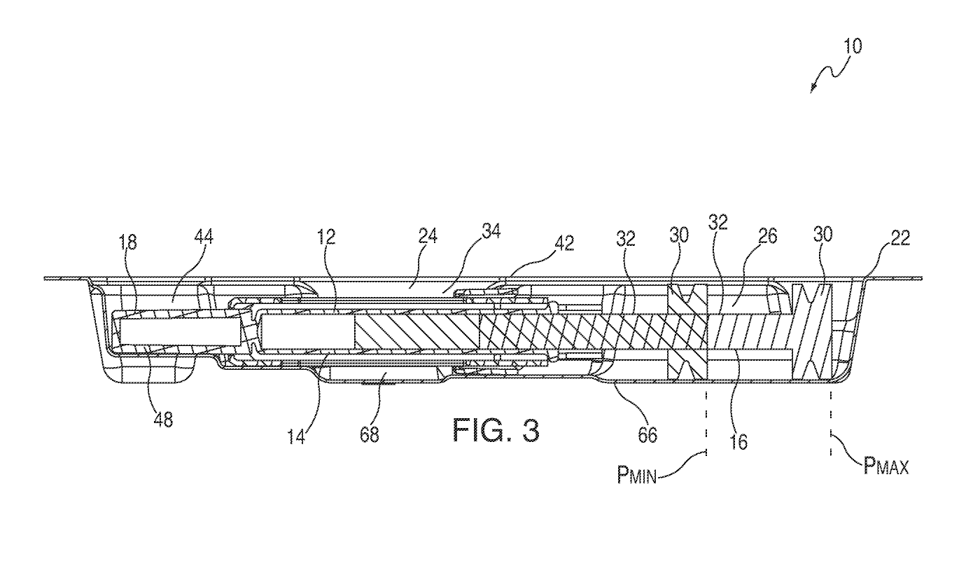

Referring now to FIGS. 1 and 3, a bottom surface 66 of the housing 22 has a profile configured for following a contour of the syringe 12. More specifically, the bottom surface 66 of the housing 22 has a first profile following the contour of the head portion 30 of the plunger 16 and the flange portion 42 of the barrel 14. Further, the bottom surface 66 of the housing 22 has a second profile following the contour of the needle end 18 of the syringe 12 and the cover 48 of the needle end. A grasping space 68 is provided in the second cavity 34 below the barrel 14 of the syringe 12 when the syringe is inserted into the chamber 24.

While a particular embodiment of the present packaging unit has been described herein, it will be appreciated by those skilled in the art that changes and modifications may be made thereto without departing from the present disclosure in its broader aspects, and as set forth in the following claims.

What is claimed is:

1. A packaging unit for accommodating a syringe having a barrel configured for slidingly receiving a plunger at one end, and having an opposite needle end, the unit comprising:
a housing defining a chamber configured for receiving the syringe, and including:
a first cavity configured for receiving the syringe plunger, and having a first set of side walls for directly engaging a head portion of the plunger without engaging a middle portion of the plunger;
a second cavity configured for receiving the syringe barrel, and having a set of upper walls for directly engaging an upper surface of a flange portion of the barrel; and
a third cavity configured for receiving the needle end of the syringe, and having a second set of side walls for directly engaging a cover configured for enclosing the needle end,
wherein the second cavity is in fluid communication with the first cavity at one end, and at an opposite end with the third cavity.

2. The packaging unit of claim 1, wherein the first cavity is configured for accommodating a plurality of displacement positions for the syringe plunger relative to the syringe barrel.

3. The packaging unit of claim 1, wherein a height of the first set of side walls in the first cavity is shorter than that of the chamber of the housing.

4. The packaging unit of claim 1, wherein a height of the first set of side walls in the first cavity is shorter than that of the second set of side walls in the third cavity.

5. The packaging unit of claim 1, wherein a pair of first feet, each first foot being disposed adjacent a corresponding one of the first side walls, and a pair of third feet, each third foot being disposed adjacent a corresponding one of the second side walls.

6. The packaging unit of claim 1, wherein a volume of the second cavity is larger than that of each of the first cavity and the third cavity.

7. The packaging unit of claim 1, wherein an axial length of the second cavity is longer than that of the first and third cavities relative to a longitudinal axis of the housing.

8. The packaging unit of claim 1, wherein a medial length of the second cavity transverse to a longitudinal axis of the housing is the longest relative to the first and third cavities.

9. The packaging unit of claim 1, wherein the second and third cavities have at least two different widths relative to a longitudinal axis of the housing.

10. The packaging unit of claim 1, wherein the second cavity has a graphical indicator designed for informing a user of a proper grasping method of the syringe for removal from the housing.

11. The packaging unit of claim 1, wherein the second cavity has at least one protrusion for directly engaging an outer surface of the syringe for providing a friction fit between the housing and the syringe.

12. The packaging unit of claim 11, wherein the at least one protrusion has a hemispherical shape, the protrusion protruding into the second cavity from side walls of the housing.

13. The packaging unit of claim 1, wherein a width of the third cavity is slightly narrower than that of the cover of the needle end for providing a friction fit between the housing and the syringe.

14. The packaging unit of claim 1, wherein the third cavity is configured for receiving the needle end of the syringe and the cover of the needle end.

15. The packaging unit of claim 1, wherein a bottom surface of the housing has a profile configured for following a contour of the syringe.

16. The packaging unit of claim 15, wherein the bottom surface of the housing has a first profile following the contour of the head portion of the plunger and the flange portion of the barrel.

17. The packaging unit of claim 15, wherein the bottom surface of the housing has a second profile following the contour of the needle end of the syringe and the cover of the needle end.

18. The packaging unit of claim 1, wherein a grasping space is provided in the second cavity below the barrel of the syringe when the syringe is inserted into the chamber.

19. A packaging unit for accommodating a syringe having a barrel configured for slidingly receiving a plunger at one end, and having an opposite needle end, the unit comprising:
a housing defining a chamber configured for receiving the syringe, and including:
a first cavity configured for receiving the syringe plunger, and having a first set of side walls for directly engaging a head portion of the plunger without engaging a middle portion of the plunger;
a second cavity configured for receiving the syringe barrel, and having a set of upper walls for directly engaging an upper surface of a flange portion of the barrel; and
a third cavity configured for receiving the needle end of the syringe, and having a second set of side walls for directly engaging a cover configured for enclosing the needle end,
wherein the second cavity is in fluid communication with the first cavity at one end, and at an opposite end with the third cavity, and
wherein a bottom surface of the housing has a first profile following the contour of the head portion of the plunger and the flange portion of the barrel, and a second profile following the contour of the needle end of the syringe and the needle end.

20. A packaging unit for accommodating a syringe having a barrel configured for slidingly receiving a plunger at one end, and having an opposite needle end, the unit comprising:
a housing defining a chamber configured for receiving the syringe, and including:
a first cavity configured for receiving the syringe plunger, and having a first set of side walls for directly engaging a head portion of the plunger;
a second cavity configured for receiving the syringe barrel, and having a set of upper walls for directly engaging an upper surface of a flange portion of the barrel; and
a third cavity configured for receiving the needle end of the syringe, and having a second set of side walls for directly engaging a cover configured for enclosing the needle end,
wherein the second cavity is in fluid communication with the first cavity at one end, and at an opposite end with the third cavity, such that a volume of the second cavity is larger than that of each of the first cavity and the third cavity, and
wherein at least one side protrusion is provided in the second cavity for directly engaging an outer surface of the syringe for providing a friction fit between the housing and the syringe.
